(19) United States
(12) Patent Application Publication
(10) Pub. No.: US 2009/0209938 A1
(43) Pub. Date: Aug. 20, 2009

(54) APPARATUS AND METHOD FOR DOSING DRUG AND WIRELESS REMOTE CONTROL OF A DRUG PUMP

(75) Inventor: Matti Aalto-Setälä, Kuusisto (FI)

Correspondence Address: YOUNG & THOMPSON, 209 Madison Street, Suite 500, ALEXANDRIA, VA, 22314, US

(73) Assignee: WRISTOP TECHNOLOGIES OY, Turku (FI)

(21) Appl. No.: 12/302,924

(22) PCT Filed: May 29, 2007
(86) PCT No.: PCT/FI2007/000146

§ 371 Date: Feb. 25, 2009

Related U.S. Application Data
(60) Provisional application No. 60/808,945, filed on May 30, 2006.

(30) Foreign Application Priority Data
May 29, 2006 (EP) 06114629.6

(51) Int. Cl. A61M 5/142

(52) U.S. Cl. 604/503; 604/66

(57) ABSTRACT

The invention comprises an apparatus meant to be carried by a user for dosing a drug, a method for dosing drug and a wireless remote control for a drug pump. The apparatus comprises a drug pump (1), a computer (9), which is arranged to control the pump, control units (10) for transmitting control functions from the user to the computer, indicators (11) for transmitting information from the computer to the user, a measuring device (12), which is arranged to produce information about the physical activity of the user, means of calculating the amount of drug needed by the user based on the information produced by the measuring device.

### TECHNICAL FIELD OF THE INVENTION

The object of the invention is an apparatus and a method for dosing a drug and wireless remote control of a drug pump according to the preambles of the independent claims presented below. The invention especially refers to a new type of portable dosing apparatus for a drug, for example insulin.

### PRIOR ART

Type I diabetes is a serious metabolic syndrome, which requires treatment for the rest of one's life. The purpose of the treatment is to maintain the glucose balance of the blood as normal as possible. The blood sugar balance in diabetes patients is however far from normal and complications are hence difficult to avoid in the long run. The complications cause large costs to society and to the diabetic, since the complications become more common as the glucose balance fluctuates. It has been calculated that an improvement of only 10% in the blood sugar balance lowers the total costs of treating diabetes by 40%. Because of this any device, which improves and facilitates the blood sugar balance is significant to diabetics and society.

Many external factors affect the blood sugar balance, of which the most central is in addition to the amount of energy received by the organism, the amount of exercise and general activity. In a diabetic, the normal insulin regulating system of the pancreas does not work because the pancreas can no longer produce insulin. The regulation is thus wholly dependent on the external insulin injected by the diabetic himself or received by him. Insulin sensitivity is individual, as is the effect of exercise on the insulin need. Still, exercise typically increases the effect of insulin. The effect of exercise on the blood sugar balance of a type 1 diabetic is dependent on many factors, such as the efficiency and duration of the workout, the care balance, the action time and dose of the insulin injected before the exercise as well as the previous food intake. Accordingly, the blood sugar may change in many ways during the workout; most often it sinks, but it may also stay the same or even rise. In studies, the effects of exercise on the long-term balance of type 1 diabetics are conflicting. Typically, exercise improves the balance only if the fluctuations it causes in the sugar balance can be prevented as much as possible. In these cases it is worthwhile adjusting the meals and insulin doses with the help of self-monitoring of blood sugar. As many factors affect the blood sugar balance, it is often difficult for a diabetic patient to estimate his insulin need. Too large an amount of insulin can lead to a life threatening hypoglycemic shock. Too small a dose on the other hand causes hyperglycemia. When occurring repeatedly or for a long duration of time, hypoglycemic shock and hyperglycemia are extremely detrimental to health and expose to dreaded complications.

With the aid of a portable insulin pump insulin is dosed under the skin of the patient by leading the insulin from the pump through a catheter to a cannula placed under the skin. The catheter and the cannula are typically changed every three days. The use of an insulin pump removes the need for repeated injections. The insulin pump doses the insulin in a continuing flow called a basic dose, which is adapted to suit the basic needs of the patient. Additional doses, or additional boluses, can also be given according to need. The patient programs the pump to feed the appropriate basic dose and desired additional boluses with the aid of the keyboard and screen of the pump. The advantage of the insulin pump is that it imitates the insulin level achieved by a healthy pancreas more closely than injection treatment. Compared to injection treatment, the insulin dosing with a pump is effective, continuous and even.

As one of the disadvantages of current insulin pumps can be seen their relatively large size. It is difficult to meet the users' demands on water resistance and durability. The current pumps are difficult to use and their function is difficult to monitor during use, as the pump is typically placed on the hip of the patient. The current insulin pumps cannot take into account the changes in the patient's system, which may greatly affect the amount of insulin needed. The use of insulin also creates social pressure for many patients. Many do not want others to know that they use an insulin pump or take injections. A pump placed on the hip is difficult to use and monitor discreetly.

It has not been possible to adequately take into account the energy consumption of the diabetic in the treatment of the diabetic. With prior art solutions it is difficult to produce accurate estimates of the amount of additional energy the diabetic needs at a given time or the most effective administration time for the insulin injections.

Disadvantages similar to the ones mentioned above are present also in dosing apparatuses for drugs other than insulin.

### THE OBJECT AND BRIEF DESCRIPTION OF THE INVENTION

It is an object of the present invention to reduce or even eliminate the above-mentioned problems appearing in the prior art.

It is especially an aim of the present invention to achieve a solution, with which a drug pump, such as an insulin pump can be made easier to use, functionally more reliable and more versatile in its use characteristics.

In order to realize for instance the objects mentioned above, the apparatus, method and remote control according to the invention are characterized by what is presented in the characterizing parts of the enclosed independent claims.

The embodiments and advantages mentioned in this text relate, where applicable, both to the apparatus and method for dosing a drug and to the wireless remote control of a drug pump according to the invention, even though it is not always specifically mentioned.

A typical apparatus meant to be carried by the user for dosing a drug according to the invention comprises
a pump, which can be arranged to dose a drug,
a computer, which is connected by first data transmission means to the pump, which computer comprises a memory and at least one computer program to be executed in the memory, which program comprises first control means for controlling and monitoring the function of the pump—typically a computer program of this sort comprises at least means for maintaining the basic level of drug dosing,
control units for transmitting control functions from the user to the computer,
indicators for transmitting information from the computer to the user,
a measuring device, which is arranged to produce information about the physical activity of the user, which measuring device is connected to the computer by second data transmission means to transmit the information produced by the measuring device to the computer.

A typical computer program according to the invention further comprises
first calculation means, which calculate an estimation of the amount of drug needed by the user, based on the information produced by the measuring device about the physical activity of the user.

A typical wireless remote control for a drug pump according to the invention comprises
a computer, which computer comprises a memory and at least one computer program to be executed in the memory, which program comprises means of controlling and monitoring the function of the drug pump—typically a computer program of this sort comprises at least means for maintaining the basic level of drug dosing,
first wireless data transmission means for connecting the computer to the pump,
control units for transmitting control functions from the user to the computer,
indicators for transmitting information from the computer to the user,
second wireless data transmission means for connecting to the computer a measuring device arranged to produce information about the physical activity of the user, in order to transmit the information produced by the measuring device to the computer,
   and that the computer program further comprises
first calculation means for calculating the amount of drug needed by the user based on the information produced by the measuring device.

A typical method for dosing a drug according to the invention comprises at least the following steps:
at least one computer program is executed in a memory of a computer, with the aid of which program the function of a drug pump is controlled and monitored,
control functions of the pump are transmitted from a user to the computer,
information about the function of the pump is indicated from the computer to the user,
information is transmitted via first data transmission means from the computer to the drug pump,
a basic level of drug dosing is maintained with the aid of the computer program,
information about the physical activity of the user is measured with a measuring device,
information is transmitted via second data transmission means from the measuring device to the computer,
the amount of drug needed by the user is calculated with the aid of the computer program and based on the information produced by the measuring device.

At least a major part of the stages in the method according to the invention can be performed programmatically with the aid of a program code.

The invention is applicable for dosing various drugs. Examples of suitable drugs to be dosed with the aid of the invention are certain liquid drugs such as insulin, certain painkillers and asthma medicines. The invention is thus applicable in the treatment of for example diabetes, pain and asthma.

Drug pumps, such as insulin pumps are devices known as such. A typical drug pump, such as an insulin pump may include for example the following parts: shells, insulin container, catheter, cannula, pump, power supply, monitor such as an LCD-monitor, keyboard.

By a computer is in this context meant a device, in the memory of which at least one computer program can be executed. The fact that the computer program comprises control means or calculation means for performing a procedure means that the computer program has program code elements, which are arranged at execution of the program to perform said procedure or to control certain physical devices, which perform said procedure.

By data transmission means is meant devices and means known as such, e.g. transmitters, receivers, conductors, connectors, antennas, program codes, with the aid of which the data communication between the parts of the apparatus is handled. The first and second data transmission means can be wholly separate from one another, or at least partially the same device.

Maintaining the basic level of drug dosing means that the computer directs according to prior art the drug pump to pump a drug according to a pre-calculated, e.g. a computer-memorized, model.

Control units for transmitting control functions from a user to a computer stand for means, through which the user or someone else can control the function of the apparatus. These control units can for example be functions keys or buttons of the apparatus. The control units can also be voice reacting, e.g. a microphone and voice recognition means. It is also possible that the apparatus is given instructions by electronic messages coming from the outside, e.g. text messages. In that case the control units comprise means with which to receive the text message or other message and the information it contains.

Indicators for transmitting information from the computer to the user can be e.g. a monitor, such as an LCD-monitor. The indicator can also transmit information acoustically, e.g. loudspeaker or headphones. The indicator can also be some other suitable means, e.g. a device with which the apparatus is made to vibrate. It is also possible that the apparatus gives information to the outside by electronic messages, e.g. text messages. In that case the indicators comprise means with which to send a text message or other message.

By a measuring device, which is arranged to produce information about the physical activity of the user, is meant a so-called activity meter. The activity meter is meant to measure the physical movement of the user. By physical movement is meant for example changes in the position of the body and movement of the body or its parts. An activity meter according to prior art is typically based on changes in acceleration or movement measured from, for example, an acceleration sensor, inertia sensor or pressure sensor, which changes are calculated into physical activity based on algorithms known as such. The changes detected by the sensors are defined as activity or movement. A common problem for a prior art activity meter is its placement. It would be good to place the meter in connection with the body of the user, for example at the hip, so that the actual physical activity of the user can be defined more accurately. The drug pump, such as the insulin pump is a good place for the acceleration sensor, as the pump is typically on the hip.

In order to be able to differentiate the movements of the body of the user, which according to the invention are referred to as physical movement, from for example the movement of a passenger sitting still in a moving car, the computer program according to the invention can be arranged to recognize automatically the quality of the movement detected by the device. For this can be used for example an intelligent algorithm, which is arranged to detect whether the user himself is moving or whether he is in a vehicle. The movement is different depending on what the user does. For example when the measuring element is an acceleration sensor, it measures changes in the movement i.e. speed, i.e. the acceleration. For example when walking the acceleration values vary within a certain frequency interval and order of magnitude. For example when driving the movement is however quite even and the changes in acceleration take place on a smaller frequency. Acceleration can be measured in a so-called triaxial manner, whereby the discrimination of different forms of movement improves.

In an embodiment of the invention the invention comprises a second measuring device, which is arranged to produce information about the physical state of the user, for example a pulse meter. The second measuring device can also be for example a glucose sensor, which produces information about the blood sugar values of the user. The glucose sensor can be a measuring device or analyzer, which is attached partly or wholly under the skin or on the skin surface. The second measuring device can also be for example a thermometer, which is arranged to measure the body temperature of the user or the external temperature. In an embodiment of the invention the measurement information produced by one or several other measuring devices is arranged to be transmitted to be used by the computer program to calculate the amount of drug needed by the user. The other measuring devices can be arranged to produce measuring results in a continuous manner.

In an embodiment of the invention the activity meter comprises two or more sensors, which produce information for the activity meter, such as acceleration sensors, inertia sensors or pressure sensors. With the aid of two or more sensors placed on different locations on the user, the activity, for example the energy consumption of the user can be measured accurately.

The means for calculating the amount of drug needed by the user based on the information produced by the measuring device about the physical activity of the user is typically an algorithm, with the aid of which the need for drug can be estimated.

Now it has thus surprisingly been established that when a device measuring the physical activity of the user, i.e. a so-called activity meter, is connected to a drug pump, surprising advantages are achieved when controlling the dosing of the drug pump. For example the energy consumption of the user is a significant factor influencing the need for numerous different drugs. With the aid of the invention the physical activity of the user can in a new effective and accurate way be taken into account when determining the dosing of the drug. For example in the defining of the insulin need of a diabetic, a new kind of efficiency and accuracy can be achieved with the aid of the invention.

One advantage of the invention is that by means of it, additional functions that will greatly benefit the user can be brought to the drug pump and the pump control.

The additional functions can for example be the same as are already known in the pulse meters of prior art. The characteristic of estimating the energy consumption known in pulse and activity meters is for example especially helpful for the user.

One advantage of the invention is that the measuring device which measures the physical activity of the user, i.e. the activity meter, can be arranged to automatically calculate a change in the drug need of the user. It is possible to set the apparatus to give an advance warning about the change in the need for drug. The invention can direct the user to make the necessary changes in the dosing of the drug. This way for example harmful fluctuations in the blood sugar balance of a diabetic can be prevented. It is even possible to program the necessary changes in the drug dosing to happen entirely automatically.

One advantage of the invention is that by means of it an estimate of the need for a drug, e.g. insulin, is obtained non-invasively. By means of the invention an estimate is obtained for example of the blood sugar value of the diabetic by measuring a so-called secondary variable, the activity of the user. With the aid of the invention it is for example possible to rarefy the need to measure the blood sugar of a diabetic from a blood sample.

One advantage of the invention is that by means of it the changes in dosing of some drugs can be estimated in new ways. For example with the aid of the activity meter one can estimate and take into account when dosing the time of the so-called post consumption of the diabetic, i.e. the fall in the need for insulin caused by workout some time after the exercise. The device according to the invention can be arranged to notify the user of the changed need for insulin a certain time after the workout.

In an embodiment of the invention, the computer program of the apparatus comprises
second calculation means for comparing the calculated amount of drug needed by the user and the amount of drug actually dosed by the drug pump and calculating the difference between these amounts, and
third calculation means for comparing said difference to a certain predetermined limiting value.

In that case the computer program typically comprises an algorithm, in which one of the variables is the information received from the activity meter measuring the physical activity of the user, such as the energy consumption of the user during a certain time interval. In addition, a variable can be for example the real-time pulse of the user. The algorithm can use as its variables for instance the user's carbohydrate intake, amount and quality of exercise and the amount and time of the dosing of a drug, for example the insulin dosing. The computer typically constantly calculates, with the aid of the algorithm, an estimation of the amount of drug needed by the user and compares this to the amount of drug fed by the pump. The received difference is compared to a limiting value pre-entered into the computer. If the limiting value is exceeded, the computer can be programmed to give the user an alarm. The alarm may for instance be a signal or the vibration of the apparatus. The alarm can also be given in the form of a text message or e-mail to some predetermined recipient. The alarm directs the user to change his drug dosing. The computer program can also comprise calculation means for calculating the energy consumption of the user based on the information produced by the measuring device.

Typically, the user has to calibrate the device before use. In that case, certain stress tests are performed with the device. The stress tests are used to simulate the workouts usually performed by the user. The necessary traced values, such as the blood sugar value for a diabetic, are measured typically at least before and after each simulated workout. The results of the simulation, i.e. the values received from the device measuring the physical state of the user and the changes in the traced values, for example changes in blood sugar, related to these values, are stored in the device. When the device during normal use discovers a change happening, similar to that of the previously simulated exercise, it is able to estimate the impact of the current workout on the traced values, for example blood sugar values of the user.

According to an embodiment of the invention, the apparatus comprises a remote control separate from the drug pump, in which remote control at least said computer, control units and indicators are placed. One of the greatest advantages of this solution is that the drug pump can be made remarkably smaller when the monitor, keyboard and the electronics connected to these are placed for instance in a wrist computer. At the same time water resistance problems are possible to solve even more easily. A small device is more comfortable for the user to carry. The remote control according to the invention is preferably connected to the activity meter and/or the insulin pump via a wireless connection.

One of the advantages of an apparatus equipped with a remote control is that by means of it usability of the drug pump improves. With the aid of the remote control the programming and monitoring of the pump is remarkably easy, compared to the use of a device placed for example on the hip. From a remote control placed for instance on the wrist, the user can easily make sure that the pump works and check how much drug has been pumped. From the device it is easy to detect for example how much drug is left, what the charge level of the pump batteries is and the estimated sufficiency time of the batteries. From the remote control it is easy to dose an additional bolus or change the basic dosing.

One of the advantages of an apparatus equipped with a remote control is that connecting a separate remote control to a computer is easy. Thus, for example programming and updating of the device or downloading of information from the remote control to the computer memory can be performed without having to detach the drug pump from the user.

One of the advantages of an apparatus equipped with a remote control is that the user's drug pump is easy to teleoperate. For instance if a child suffering from diabetes is sleeping is his own room, his mother or father can, when necessary, check from another room that the pump is working. The parents can in this way monitor the insulin supply and for example activity of the child sleeping in a separate room. The device can be set to alarm if the child has been too inactive for a certain period of time. An unnaturally long period of immobility can have been caused by for instance hypoglycemia.

With the aid of an apparatus equipped with a remote control the social pressure some users feel for using a drug pump is diminished. The remote control is always with the user, discreet and easy to use. The remote control can be incorporated in a fashionable and sporty pulse or activity meter. In this way, the drug pump, which makes some users ashamed, irritated or afraid, turns into an interesting exercise or sports computer. The design or features of the devices are easy to adapt so that wrist devices in their own style are made for different user groups, For instance different models can be made for a child, an athlete and a senior. Basic functions when it comes to interacting with the drug pump can always be kept the same.

In an embodiment of the invention the data transmission means between the computer and the measuring device according to the invention of the drug pump is wireless. The means can use for example the 2.4 GHz frequency, known from pulse meters.

In an embodiment of the invention the drug pump and the measuring device are situated in the same unit, separate from the remote control. For example the acceleration sensor of an activity meter can be arranged in the same device as the drug pump, which device is attached to the user. In this way, the measuring device according to the invention is always with the user, e.g. on his hip. In that case, no prior art pulse belt or other particular sensors or means of attaching sensors are needed. In this way the comfort of use is improved.

In an embodiment of the invention the remote control is a wrist computer. Typically the remote control then comprises attaching means for attaching it in a removable manner to the wrist of the user, for instance a wristband. The length of the attaching means is normally adjustable, typically between 100-200 mm. A remote control attached to the wrist is easy to use and discreet to carry along.

In an embodiment of the invention the drug pump, activity meter and computer are in one and the same device, meant to be carried by the user. In an embodiment all the parts of the device are in one and the same device. In an embodiment at least the drug pump, computer and the computer programs in its memory, the first data transmission means, control units for transmitting control functions from the user to the computer, indicators for transmitting information from the computer to the user, the activity meter and second data transmission means are situated in one and the same device. When attached for example to the hip or another suitable location a one-piece device such as this measures the physical activity of the user from a suitable place.

In an embodiment of the invention the measuring device comprises a pulse meter, which can be arranged to measure the pulse of the user. A typical prior art pulse meter is based on two electrodes placed on the skin, where the pulse is calculated based on the tension difference between the electrodes. Usually the pulse meter includes a pulse belt attached to the skin, in which belt the sensors are situated. A typical pulse belt sends the pulse information wirelessly to some terminal or stores the pulse information in its own memory. It has been attempted to measure the pulse from for instance the wrist, usually with poor results. The reason for this is that a typical pulse sensor needs to be attached to the skin and a wrist device easily moves when the user moves. The drug pump, such as an insulin pump, however, is usually steadily attached to the skin, whereby it is possible to place into the pump the two sensors needed for measuring the pulse.

In an embodiment of the invention the apparatus is arranged to collect and store information for example about the movement of the user, the dosing of the drug, the physical variables such as pulse or blood sugar concentration. When information such as this is stored as a function of time, versatile statistics can be collected with the device. This kind of information can be utilized for example when drawing up exercise and nutrition recommendations for a patient. The dosing or quality of the drug can also when necessary be corrected based on the statistics.

In an embodiment of the invention the apparatus is arranged to collect and store information, for example about the movement of the user, the dosing of the drug, or the physical variables such as pulse, especially when the user is sleeping. From this information the sleeping habits of the user can be discovered. For example the sleeping habits or changes in them, the activity level of the user when sleeping or the need for insulin during sleep can be tracked. The device may have a specific function, which is activated when the user goes to bed. The device may be arranged to recognize the sleep of the user for example by the user's movement or physical variables such as the pulse.

In an embodiment of the invention the apparatus is arranged to produce information for the user about his or her movement either in real time or to store information in the electronic memory for later use. The activity meter can be arranged to produce information for example on the speed of the user at a given time, the distance he or she has traveled, his or her average speed or energy consumption.

In an embodiment of the invention, the measuring device comprises a blood sugar meter, which is arranged to measure the blood sugar of the user. The meter can be arranged in connection with the drug pump or the remote control, such as a wrist device. The meter can also be arranged in connection with a cannula placed under the skin, whereby the blood sugar measuring can be performed through this blood contact. In an embodiment of the invention the blood sugar can be measured for instance with the aid of light from the earlobe or fingertip, i.e. the measuring can thereby be performed without blood.

The device according to the invention can be arranged to determine for the user suitable measuring times for blood sugar based on the activity of the user. If the device according to the invention comprises a blood sugar measuring device, the device can be arranged to produce an alarm in case the real-time change in the blood sugar value does not correspond to the expected change in blood sugar value, pre-calculated based on the activity of the user and the dosed amount of insulin.

In an embodiment of the invention, the computer program comprises one or more of the following
third control means for giving an additional dose of drug,
fourth control means for making a change in the basic dosing level of the drug.

Thereby, the user can, for example, by pressing the function keys of the remote control give an additional dose of drug when desired or make a change in the basic dosing level of the drug dosed by the device. An additional drug dose may be needed for example in connection with a diabetic's eating. Momentary changes in the basic dosing level of many drugs can be needed for example in connection with increased exercise.

In an embodiment of the invention, the apparatus comprises safety means to prevent incorrect dosing. In an embodiment of the invention, the amount of the additional bolus can be programmed so that a drug, such as insulin can in no case be given in excess of a predetermined limiting value. Typically, the user can program certain maximum values into the device, which values the dosing cannot exceed. In an embodiment of the invention, the apparatus has a keylock. The keylock may be mechanic or electronic. It is also possible that the apparatus has both a mechanic and an electronic keylock at the same time. For example the remote control can demand a PIN code or a similar code before the dosing can be changed.

In an embodiment of the invention, one or more of the following are indicated to the user for example on the LCD monitor of the remote control:
An indicator arranged to move when the pump is functioning. This may for example be an indicator programmatically realized on the monitor of the remote control, which indicator always moves when the drug pump is functioning. In this way the user can with a quick glance assure himself of the function of the device.
Charge level of the pump batteries displayed for example as a 4-8-level column display.
Remaining estimated sufficiency time of the batteries displayed as a numerical value, for example as hours and minutes.
Amount of remaining drug displayed for example as a 4-8-level column display.
Remaining estimated sufficiency time of the drug displayed as a numerical value, for example as hours and minutes. The sufficiency time can be estimated for example based on the preceding consumption in the same way that on-board computers in cars typically evaluate the adequacy of the fuel.

All the above-mentioned things can be presented in many different ways. The manner of representation can among others be a so-called column indicator or a dial indicator. The values can also be expressed as numbers.

In an embodiment of the invention, there are third data transmission means, for example suitable interfaces for connecting the computer of the apparatus according to the invention to a second computer. In that case the computer program typically comprises also forth data transmission means to perform data transmission between the computer and the second computer. For example a device according to the invention, such as a wrist device could be connected via a USB cable to the computer or the data transmission can be realized wirelessly for example with the aid of Bluetooth™ technology. Among others the following information, which affects the estimation of the need for drug, for example the need for insulin, can be entered from another computer into the computer of the apparatus for the purpose of the algorithm:
Nutrition plan of the user.
An estimation of the amount of sleep of the user. This can also be calculated from the activity or pulse of the user.
Certain illnesses the user has.
Stress level of the user, which can also be estimated for example directly from the pulse.
Glucose database of the user's blood.

Third and fourth data transmission means can be arranged to send information stored in the computer of the device according to the invention, such as information gathered on the sleeping habits of the user, to another computer for example for a more thorough analysis.

The third and fourth data transmission means can be compatible with the GSM or another mobile phone network. In that case information can be sent from the device according to the invention to places situated even far away, for example to the computer of the health clinic, the hospital or other health service provider. Data transmission compatible with the GSM or other mobile phone network is well suited for sending an alarm to a desired destination for example when the device detects an overly large drug dosing or a blood sugar content, which is too low.

In an embodiment of the invention the device according to the invention comprises satellite positioning means, for example a GPS locator (Global Positioning System). The positioning means can be used for example to give the location of the user when the device gives an alarm because of an overly large drug dosing or a blood sugar content of the user, which is too low or too high.

In an embodiment of the invention the memory of the computer of the device can have a computer program to be executed, with the aid of which the user can input information about the nutrition he or she has consumed. The computer can have a database ready, wherefrom the consumed meal can be chosen, whereby the database has ready information about the nutrition content in question. The computer program can also allow the user to input manually information about his or her consumed nutrition. Possible information, which the computer program according to the invention utilizes to calculate the necessary drug amount may for example be the amount of energy, carbohydrates or fat contained in the food.

A portable remote control according to the invention preferably weighs less than 300 or less than 200 grams, for example 40-100 grams. A remote control according to the invention has for instance the following size: width 40-60 mm, length 40-70 mm and thickness 10-20 mm. The above-mentioned measurements concern the frame of the remote control. The size of additional parts, such as a wristband, which are possibly attached to the frame, varies according to need.

The part of the apparatus according to the invention, which comprises the drug pump, i.e. the so-called pump section preferably weighs less than 200 or less than 100 grams, for example 50-150 grams. The pump section according to the invention has for instance the following size: width 40-100 mm, length 40-100 mm and thickness 10-30 mm. The above-mentioned measurements concern the frame of the pump section. The size of additional parts possibly attached to the frame section, such as attaching means, with which the pump section is attached to the user, varies according to need.

The volume flow pumped by the drug pump, for example the insulin pump, can for example be 200 microliters-1.5 milliliters per hour.

A possible device meant to be carried by the user comprises
a computer, which comprises a memory and at least one computer program meant to be executed in the memory,
control units for transmitting control functions from the user to the computer,
indicators for transmitting information from the computer to the user,
a measuring device, i.e. an activity meter, which is arranged to produce information about the physical activity of the user, which measuring device is connected to the computer by data transmission means to transmit the information produced by the measuring device to the computer,
   whereby the computer program comprises
program code elements, which are arranged to produce a parameter describing the activity of the user, which can be presented to the user with indicators.

In an embodiment of the invention the computer program comprises program code elements, which are arranged to assess, based on the mentioned parameter describing the activity and other information in the memory of the computer, a certain physical state of the user, such as the glucose value of the blood. It is possible that the computer program is also arranged to assess further the drug dosing needed by the user, for example to calculate an estimate of the amount of drug needed by the user. It is possible that the computer program is arranged to produce a reminder for the user, when the user according to the computer program needs for example additional energy or a drug dose, for example an insulin injection. In such a portable device there is thus typically not a drug pump. In one such an embodiment all the above-mentioned parts are situated in a wrist device.

In an embodiment a portable wrist device without a drug pump is arranged to aid the diabetic with the need for insulin injections. The acceleration sensor or corresponding at the wrist calculates the energy consumption of the user and reveals it to the diabetic. The device can further be arranged to assess and give information to the diabetic about the times, when the user needs an insulin injection or additional energy. Such a device can thus be used as an instrument in a diabetic's multi injection treatment as a meter of the energy consumption and/or insulin need.

### BRIEF DESCRIPTION OF THE FIGURES

The invention is described in more detail below with reference to the enclosed schematic drawing, in which

FIG. 1 shows one apparatus according to the invention,

FIG. 2 shows an enlargement of the remote control monitor seen in FIG. 1,

FIG. 3 shows one method according to the invention as a flow chart, and

FIG. 4 shows one method according to the invention as a flow chart.

### DETAILED DESCRIPTION OF THE EXAMPLES OF THE FIGURES

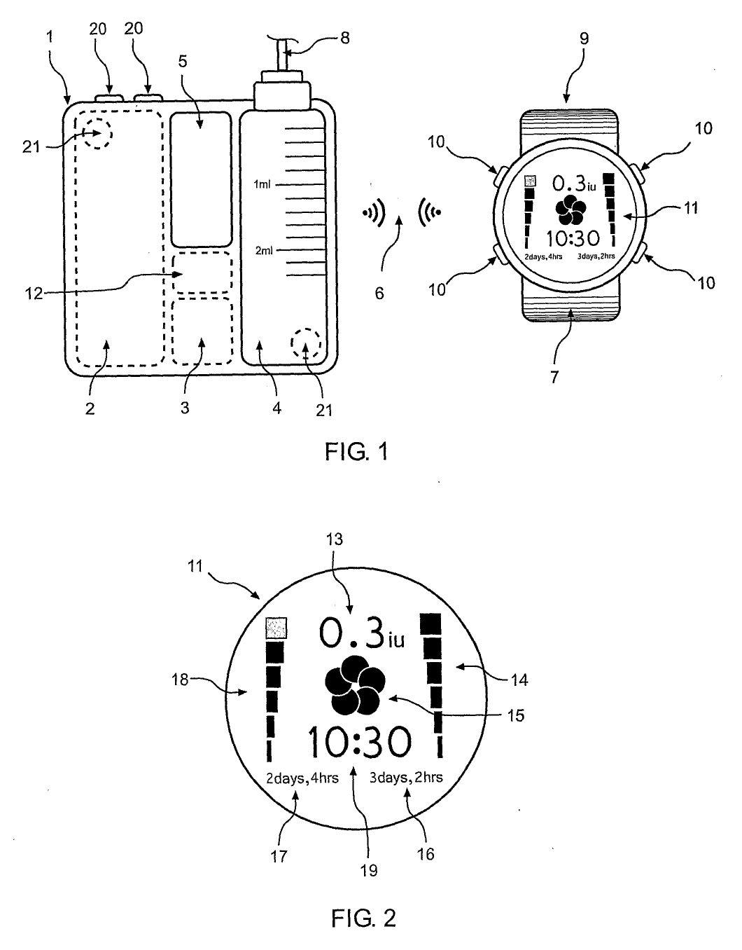

FIG. 1 shows one apparatus for dosing insulin according to the invention, intended to be carried by a user. An apparatus as the one shown would be suitable also for the dosing of many other drugs.

The apparatus has two independent parts, which in this text will be referred to as a pump section 1 and a wrist device 9. The pump section 1 is meant to be attached to the hip of the user. The pump section has its own power supply, e.g. a battery 2. A wireless transmitter and receiver are marked with number 3 and an LCD monitor with number 5. From an insulin container 4 the pump (not shown) doses insulin into a catheter 8. There is also an activity meter 12 in the pump section. Part 12 can also comprise for example a pulse meter. Reference number 21 is used to mark possible sensors of the pulse meter. The functions of the pump section can be controlled with function keys 20.

The wrist device 9 is attached to the arm of the user with a wristband 7. The wrist device as well as the pump can be controlled with function keys 10. A monitor 11 shows the user the variables related to the function of the pump. The wireless transmission between the pump section 1 and the wrist device 9 is shown schematically with symbols 6. The pump section can be attached to the user by for example a particular belt, in the same way as prior art pulse meters. The pump can also be placed in a belt case or other clothing accessory worn by the user.

FIG. 2 shows in more detail the monitor 11 of the wrist device. On the left is shown with a column display 18 the amount of insulin still available in the container 4. A numerical display 17 shows a time estimate of the adequacy of insulin. This estimation is obtained by dividing the amount of insulin in the container with the consumption of insulin from a period at a certain point in the past. On the right edge of the screen is shown with a column display the charge level of the batteries. The numerical display 17 shows a time estimate of the adequacy of the batteries. A numerical display 13 tells of the current size of insulin dosing. A numerical display 19 shows the time.

Insulin pumps sometimes fail, e.g. they can block up. An even and continuous dosing of insulin is often of primary importance to the user. Often users need to check the function of the device on a regular basis. In the pump section 1 there is a sensor (not shown), which monitors the function of the pump. Between the numbers 13 and 19, approximately in the centre of the monitor 11 is a symbol 15. The sensor monitoring the pump gives the computer information about whether the pump is functioning. The computer has program code elements, which control the symbol 15 shown on the monitor 11 according to the information received from the sensor. The symbol 15 is arranged to be mobile, e.g. rotating, when the pump is working and pumping insulin in a normal manner. In this way the user notices with a glance that the pump is working.

The wrist device 11 can be used as a normal wrist watch. For this purpose, basic functions of a watch such as time, alarm and calendar with alarm are programmed into it. In addition the wrist device 11 can have a complete calorie table with selectable courses from a computer database and calorie calculation. From a calorie table the user can easily choose the courses he has eaten. The computer program according to the invention can be arranged to take into consideration the chosen foods immediately when estimating the need for insulin with the aid of the algorithm according to the invention.

When the device according to the invention detects a clear change in the measured physical activity of the user, for example in the amount of exercise, it recommends a change in the dosing. The device can ask for the estimated duration of the rest or exercise. The device can then make a recommendation based on the reply, which recommendation the user acknowledges as accepted.

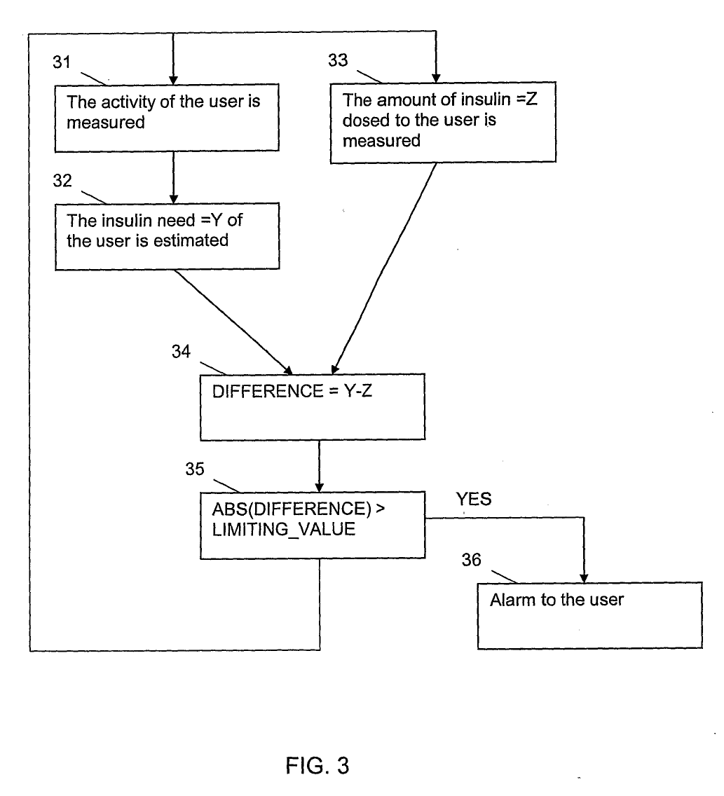

FIG. 3 shows in a simplified way as a flow chart one embodiment of the method according to the invention. In stage 31 the activity of the user is measured. In stage 32 the insulin need Y of the user is estimated using the activity measured in stage 31. At the same time in stage 33 the actual insulin amount Z dosed by the insulin pump is constantly measured. The difference between Y and Z is formulated in stage 34. In stage 35 the absolute value of the difference is compared to a predetermined limiting value. If the difference is greater than the limiting value, the device gives the user an alarm 36 and encourages him to change the dosing of the insulin. The measurings in stages 31 and 33 are constantly continuing.

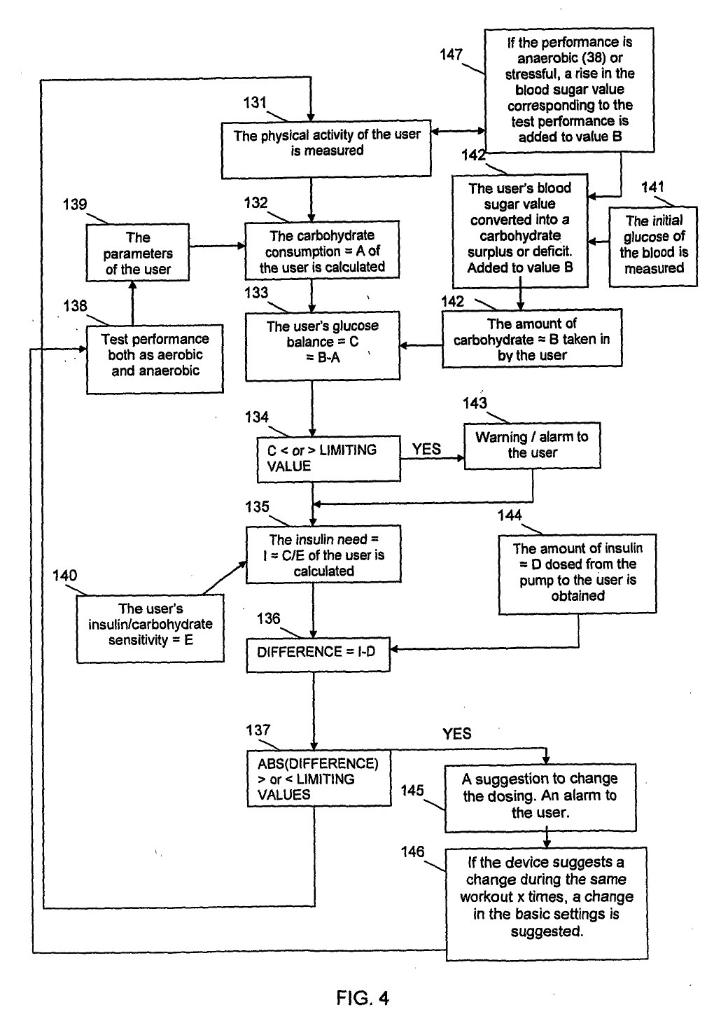

FIG. 4 shows in a simplified way as a flow chart another embodiment of the method according to the invention. In stage 131 the physical activity of the user is measured for example by measuring the acceleration or the pulse. In stage 132 the energy/carbohydrate consumption is derived from the activity. The correlation between the activity and the energy consumption is obtained from a test run performed by the user. In stage 133 the energy balance C of the diabetic is obtained by deducting the consumed energy A from the energy B released from the carbohydrate reserves. In stage 134 the energy reserves C of the user are monitored, i.e. the carbohydrate reserves are compared to certain limiting values. For example if the energy reserves threaten to diminish, the user is in stage 143 advised to eat or drink or change the insulin dosage. The diminishing of the reserves also expresses itself as a decline in the blood glucose level. In stage 135 the insulin need I is calculated by dividing the energy balance C with the insulin sensitivity E of the diabetic. In stage 136 the difference between the insulin need and the dosed insulin is calculated. In stage 137 is studied if the difference is larger or smaller than the limiting values. If it is, a change in the insulin dosing is suggested to the user. In stage 138 the test performance is performed at regular intervals and it is used to ensure that the right coefficients are used in the calculation when calculating the energy consumption and the need for insulin. The test performance can be performed for example so that the same kind of exercise, which is as easy to measure as possible, is done at all times, such as running, and the blood sugar value is measured before and after the run. The test performance can also be any workout the user normally practices. The device automatically recognizes changes in the activity of a normal day by making a continuous log of the activity and comparing it again to the measured information. The test performance can also be performed so that the anaerobic threshold is exceeded, whereby the blood glucose content possibly rises. A stressful performance, e.g. in a competitive situation, has the same effect. In other words, default values in the sports the user practices are created based on the test performance for the changes in insulin need for each tested performance and stress level. By parameters in stage 139 are meant the personal coefficients of the user, obtainable from the test performance, which coefficients are used when calculating the energy consumption and with the aid of which the device recognizes changes in the activity. A certain carbohydrate consumption is thus established for the device based on a certain performance. In stage 140 the individual insulin sensitivity E of the user is calculated with a widely known formula, which insulin sensitivity determines how much insulin is needed to level out the elevating effect of a certain amount of carbohydrate on the blood glucose content. Later the device learns or the device is told the own real sensitivity values determined by use and the test performances. In stage 141 the blood glucose content of the user is measured. In stage 142 the user inputs into the device the amount of carbohydrate consumed. The user's blood sugar value converted into a carbohydrate surplus or deficit is added to the value B. In stage 143 the user is warned about a danger situation. In stage 144 real-time information about the dosed amount of insulin and possibly the glucose level is automatically received to the insulin pump via a wireless connection. In stage 145 the device suggests to the user a change in the dosing. In stage 146, if the device during the same workout suggests a change a certain amount of times, a change in the basic settings is suggested. In stage 147, if the performance is anaerobic (stage 138) or stressing, a rise in the blood sugar value corresponding to the test performance is added to the value B.

Figures show only a few preferred embodiments according to the invention. The Figures do not separately show matters that are irrelevant in view of the main idea of the invention, known as such or obvious as such for a person skilled in the art. It is apparent to a person skilled in the art that the invention is not limited exclusively to the examples described above, but that the invention can vary within the scope of the claims presented below. The dependent claims present some possible embodiments of the invention, and they are not to be considered to restrict the scope of protection of the invention as such.

1-23. (canceled)

24. An apparatus meant to be carried by a user for dosing insulin, which apparatus comprises
a pump, which can be arranged to dose insulin,
a computer, which is connected by first data transmission means to the pump, which computer comprises a memory and at least one computer program to be executed in the memory, which program comprises first control means for controlling and monitoring the function of the pump,
control units for transmitting control functions from the user to the computer,
indicators for transmitting information from the computer to the user,
a measuring device, which is arranged to produce information about the physical activity of the user, which measuring device is connected to the computer by second data transmission means to transmit the information produced by the measuring device to the computer,
and the computer program comprises
first calculation means for calculating the amount of insulin needed by the user based on the information produced by the measuring device
wherein the apparatus is arrangeable at hip of the user, and the measuring device comprises an acceleration sensor for measuring changes in the physical movement of the user.

25. An apparatus according to claim 24, wherein the apparatus comprises a remote control separate from the pump, in which remote control at least said computer, control units and indicators are placed.

26. An apparatus according to claim 24, wherein the computer program comprises
second calculation means for comparing the calculated amount of insulin needed by the user and the amount of insulin actually dosed by the pump and calculating the difference between these amounts, and
third calculation means for comparing said difference to a certain predetermined limiting value.

27. An apparatus according to claim 26, wherein the computer program comprises alarm means for indicating an alarm with the indicators, if the difference exceeds said predetermined limiting value.

28. An apparatus according to claim 26, wherein the computer program comprises second control means for changing the insulin dosing, if the difference exceeds said predetermined limiting value.

29. An apparatus according to claim 24, wherein the first and/or second data transmission means are wireless.

30. An apparatus according to claim 24, wherein the pump and the measuring device are placed in the same unit, separate from the remote control.

31. An apparatus according to claim 24, wherein the remote control is a wrist computer and comprises attaching means for removably attaching it to the wrist of the user.

32. An apparatus according to claim 24, wherein at least the pump, the measuring device and the computer are in one and the same device, meant to be carried by the user.

33. An apparatus according to claim 24, wherein the measuring device comprises a pulse meter, which can be arranged to measure the pulse of the user.

34. An apparatus according to claim 24, wherein the apparatus is arranged to collect and store in the memory of the computer information about the movement, insulin dosing or physical variables such as pulse of the user.

35. An apparatus according to claim 24, wherein the computer program comprises fourth calculation means for calculating energy consumption of the user based on the information produced by the measuring device about the activity of the user.

36. An apparatus according to claim 24, wherein the indicator is arranged to indicate one or more of the following:
an indicator arranged to move when the pump is functioning,
charge level of the batteries of the pump,
amount of the remaining insulin,
estimated sufficiency time of the remaining insulin.

37. An apparatus according to claim 24, wherein the computer program comprises one or more of the following:
third control means for giving an additional dose of insulin,
fourth control means for making a change in the basic insulin level,
and the control units are arranged so that with them the user can activate one or more of the above-mentioned control means, if desired.

38. An apparatus according to claim 24, wherein the apparatus comprises third data transmission means for connecting the computer to a second computer and the computer program comprises fourth data transmission means for performing data transmission between the computer and the second computer.

39. An apparatus according to claim 24, wherein the apparatus comprises a glucose sensor, which is arranged to produce information about the blood sugar values of the user, and data transmission means for transmitting the information produced by the glucose sensor to the computer.

40. An apparatus according to claim 24, wherein the measuring device comprises an inertia sensor or a pressure sensor.

41. A wireless remote control for an insulin pump, wherein the remote control comprises
a computer, which computer comprises a memory and at least one computer program to be executed in the memory, which computer program comprises first control means for controlling and monitoring the function of the pump,
first wireless data transmission means for connecting the computer to the pump,
control units for transmitting control functions from the user to the computer,
indicators for transmitting information from the computer to the user,
second wireless data transmission means for connecting to the computer a measuring device comprising an acceleration sensor, the measuring device being arranged to produce information about the physical activity of the user, in order to transmit the information produced by the measuring device to the computer,
and the computer program comprises
first calculation means for calculating the amount of insulin needed by the user based on the information produced by the measuring device.

42. A method for dosing insulin, in which method
at least one computer program is executed in a memory of a computer, with the aid of which program the function of an insulin pump is controlled and monitored,
control functions of the pump are transmitted from a user to the computer,
information about the function of the pump is indicated from the computer to the user,
information is transmitted via first data transmission means from the computer to the pump,
a basic level of insulin dosing is maintained with the aid of the computer program,
information about the physical activity of the user is measured with a measuring device comprising an acceleration sensor,
information is transmitted via second data transmission means from the measuring device to the computer, and
the amount of insulin needed by the user is calculated with the aid of the computer program and based on the information produced by the measuring device.

43. A method according to claim 42, wherein a remote control is used, in which remote control said computer memory is located and with the aid of which
the user gives control functions to the pump,
information about the function of the pump is indicated to the user, and
information is transmitted wirelessly via the first and second data transmission means.

44. A method according to claim 42, wherein
the amount of insulin needed by the user and the amount of insulin actually dosed by the pump are calculated, and the difference between these is calculated, and
the absolute value of said difference is compared to a certain predetermined limiting value.

45. A method according to claim 44, wherein an alarm is indicated to the user if the absolute value of the difference exceeds said predetermined limiting value.

46. A method according to claim 44, wherein the dosing of the insulin is changed if the absolute value of the difference exceeds said predetermined limiting value.
